(19) United States
(12) Patent Application Publication
(10) Pub. No.: US 2005/0256379 A1
(43) Pub. Date: Nov. 17, 2005

(54) Systems and methods for remote touch-dependent evaluation

(76) Inventors: Yvedt Matory, Dedham, MA (US); Randall Kennedy, Dedham, MA (US)

Correspondence Address: GREENBERG TRAURIG, LLP, ONE INTERNATIONAL PLACE, 20th FL, ATTN: PATENT ADMINISTRATOR, BOSTON, MA, 02110, US

(21) Appl. No.: 11/126,844

(22) Filed: May 10, 2005

Related U.S. Application Data
(60) Provisional application No. 60/570,527, filed on May 12, 2004.

(51) Int. Cl. A61B 5/00

(52) U.S. Cl. 600/300

(57) ABSTRACT

Techniques for use in a remote patient care system such as a remote patient care system that connects patients and health care professionals over a network.

### RELATED U.S. APPLICATION(S)

The present application claims priority to U.S. Provisional Application Ser. No. 60/570,527, filed May 12, 2004, which application is hereby incorporated herein by reference.

### TECHNICAL FIELD

The present invention is directed to patient evaluation, and more particularly to remote patient evaluation by touch-dependent techniques.

### BACKGROUND

Healthcare expenditures continue to represent the single largest sector of the U.S. economy, with over $1 trillion, or 14 percent, of the gross domestic product spent in 2000. Healthcare costs are at the highest level in two decades with no relief in sight. Hospitals and insurance providers are facing severe budget constraints due to shrinking reimbursements and higher costs of care.

Despite significant advances in healthcare delivery, the primary driver of controllable healthcare costs remains hospital stays. Latest figures from 1998 show that over 33% of total U.S. healthcare expenditures were a result of hospitalization (Health Affairs). Despite the strain of extended stays on care-givers, protracted time in a hospital does not necessarily translate into better quality-of-care for patients. Hospitalization can lead to significant potential medical complications, for instance, increase risks of infection, medication error, patient depression, which can further increase healthcare costs. Hospital acquired infections cost over $2 billion annually, according to a survey conducted from 1986 to 1998 by the National Nosocomial Infections Surveillance System of the CDC (eMedicine Journal). In addition, hospitalization-related medical errors lead to 98,000 deaths annually (Institute of Medicine). Prolong hospitalization can also exacerbate “bed-shortages” experienced in many hospitals.

There has been a significant drive to decrease the length of hospitalization and to develop ways of taking care of patients at home. Hospitals and insurance providers have, over the past decade, decreased length of stay through a variety of efforts, but have reached the point of diminishing returns. Specifically, traditional discharge planning procedures can often fail to provide adequate care to a patient after discharge. Often patients are left to manage their own dressings, monitor drainage, and adjust their own pain medication intake within the bounds of prescribed prescriptions. While, in some cases, a nurse will visit a patient at home to evaluate incisions, drainage, and vital signs, such visits may be abbreviated and far between.

Additionally, a patient must often coordinate his/her own care with many different providers. For example, a patient must often schedule follow-on visits with medical specialists, e.g., radiation oncologist, surgeon, and/or other specialists. Furthermore, a patient must, at times, coordinate access to emotional and psychological services, such as volunteer support, recovery aid, situational social workers, and psychiatric services supporting quality of life issues.

The establishment of more expanded care in the home can be further limited due to existing technology. Currently, technology exists to evaluate patients remotely in many ways. They include: remote heart rate, remote lung function, remote stethoscopes, remote weight scales, remote audio-visual communication. These functions, although may be useful in their own way, do not allow for touch-dependent evaluation and diagnosis of medical problems, for instance, abdominal symptoms. Whereas current technology only allows for measurements which are indirect indicators of existing medical problems, touch-dependent evaluation can be more specific and allow for a more accurate evaluation and diagnosis.

### SUMMARY OF THE INVENTION

The present invention, in one embodiment, provides a system for remote patient evaluation by touch-dependent protocol in connection with a remote patient care system, such as a remote patient care system that connects patients and health care professionals over a network using video conferencing.

In accordance with one embodiment, the system includes a computer. The system also includes a pad in communication with the computer and sufficiently sized to at least partially cover an area to be evaluated. The system further includes a plurality of sensors placed on the pad to measure characteristics of the area to be evaluated. The system can also include data transmission means connected to a public network for transmitting, in real time, measured characteristics to a remote location for evaluation. In one embodiment, the sensors may be positioned relatively close to one another to provide enhanced sensing capability and accuracy.

In accordance with another embodiment of the present invention, a method for remote touch-dependent evaluation is provided. The method includes initially providing a pad sufficiently sized to at least partially cover an area to be evaluated on a patient and having a plurality of sensors to measure characteristics of the area to be evaluated. Next, the pad may be positioned on to a body part to be evaluated. Then the sensors on the pad may be activated to initiate measurement. In an embodiment of the invention, activating the sensors may be accomplished remotely from the remote site. Thereafter, measured characteristics may be transmitted, in real time, to a remote site. The measured characteristics may subsequently be reviewed, from the remote site, for evaluation purposes.

### BRIEF DESCRIPTION OF THE DRAWINGS

FIG. 1 is a diagram of a system for remote disease management.

FIG. 2 is a diagram of a graphical user interface presented to a care provider.

FIG. 3 is a diagram of a graphical user interface presented to a patient.

FIG. 4 is a flow-chart of a process for remote disease management.

FIG. 5 is a flow-chart of a process for remote body imaging evaluation.

FIG. 6 is a flow-chart of a process for adjusting a remote disease management process.

FIG. 7 is a diagram of a computer platform suitable for adjusting protocol criteria based on collected data.

### DETAILED DESCRIPTION OF SPECIFIC EMBODIMENTS

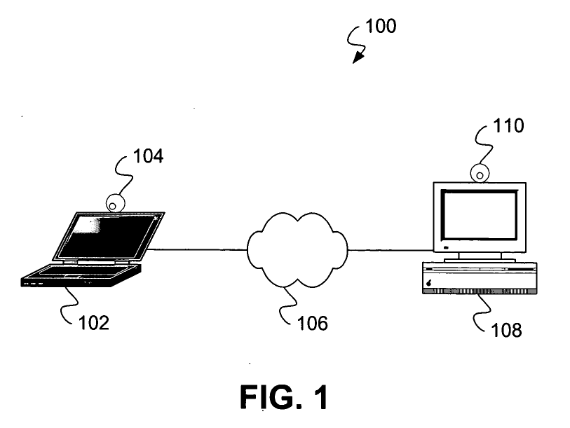

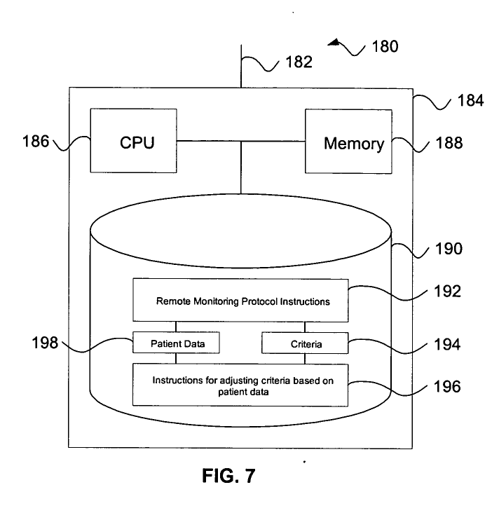

FIG. 1 shows a system 100 that enables health care professionals to remotely monitor and provide care to patients. As shown, the system 100 includes a patient's computer 102 and a health care provider's computer 108 that share data over a network 106, such as the Internet. While shown as a laptop 104, the patient's computer 102 may be a desktop model, Web TV, handheld device, wireless unit, and so forth. The system 100 may also include auxiliary computers such as an administrative computer (described in conjunction with FIG. 7).

Both patient and health care provider computers 102, 108 feature video cameras 104, 110 and microphones (not shown) for acquiring still-images, audio, and/or video data. The computers 102, 108 can communicate using network conferencing software such as Microsoft's NetMeeting or CUSeeMe. Instead of these off-the-shelf options, the computers 102, 108 may use dedicated conferencing/communication software developed for the system. Use of real-time conferencing enables health care professionals to provide patients with live interactive care without inconvenient travel to a hospital or extended time in a waiting room.

The system 100 offers an integrated approach to patient care and offers features that ensure proper treatment. For example, as explained below, the system 100 can dynamically adjust care parameters based on patient outcomes, satisfaction surveys, and other collected data. Additionally, as described below, the system 100 can provide a script for health care providers using the system 100 to maintain a high level of care.

The system 100 can enable hospitals to discharge patients earlier than traditionally contemplated while increasing the quality-of-care experienced by a patient. For example, patients can more quickly return to the personal comfort and reassurance of home. Additionally, unlike patients' discharged after a lengthy hospital stay, patients using the system 100 enjoy continued access to hospital staff.

In addition to greater patient satisfaction and improved quality-of-care, the system 100 offers cost savings to many in the health care landscape. For example, by decreasing the use of costly in-patient and out-patient resources, hospitals reduce the financial obligations of insurers and hospital networks. Additionally, remote monitoring can greatly increase the productivity of health care professionals. For example, a nurse using the system can quickly monitor many patients without leaving their chair.

The system 100 uses a number of safeguards to ensure patient confidentiality while transmitting data over the public network 106. For example, the system 100 can use standard methods of encryption such as using Secure Sockets Layer (SSL) software. To further enhance security, the system 100 independently transmits and encrypts visual, audio, text, health care metrics (e.g., vital signs), and other information. The system 100 may also make use of passcodes to enhance security. The exchange of information complies with Health Insurance Portability Accountability Act (HIPAA) regulations.

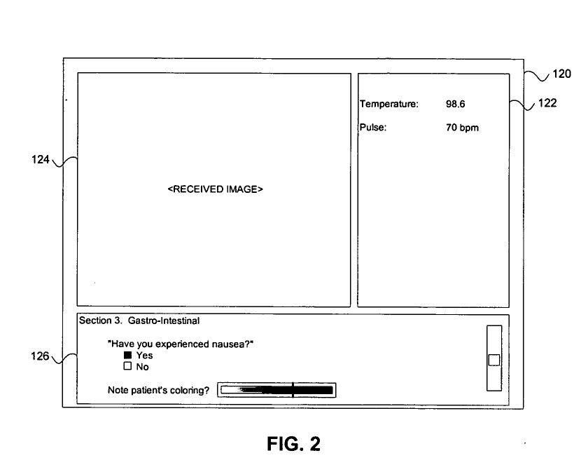

FIG. 2 shows an example of a user interface 120 presented to a health care professional during a remote care session. The interface 120 enables a professional to remotely assess patient status against care management guidelines for the patient's clinical condition. The user interface 120 includes a region 124 for viewing image/video data transmitted by the patient's computer. The user interface 120 can also present other data collected and transmitted by the patient computer. For example, the patient's computer may be equipped with sensors and other devices for collecting heart rate, blood pressure, glucose levels, spirometry, as well as depth or physical injury that may be the source of pain. The user interface 120 can dynamically update the display of these values.

The information presented by the user interface 120 enables a nurse to gauge a patient's condition, advise when a patient needs to be seen in the physician office, and alert the nurse to request other information or views of the patient. The user interface 120 may also provide controls (not shown) that enable the health professional to remotely control the patient's camera, for example, by changing its orientation and/or magnification.

As shown, the user interface 120 also presents a concurrently displayed script 126 region that provides guidance to a health care professional during a patient session. The script can remind the health care professional to ask certain questions, note particular aspects of a patient, and so forth. As shown, the script 126 can also receive data entry via familiar user interface control “widgets”, such as radio buttons, sliding scales, text boxes, and so forth. As the nurse responds to script 126 questions and prompts, the script 126 instructions can store the responses and determine the next questions/statements to present.

The particular script 126 selected for use during a remote session may depend on the particular ailment, patient, duration since last visit, and other factors. Additionally, the script 126 may incorporate conditional logic that varies the questions/prompts presented based on the patients previous responses or other collected information. For example, if the health care computer receives vital sign data indicating a quickened pulse, the script 126 logic may cause a question to be presented asking whether the patient feels feint. Similarly, as shown, if a patient reports nausea, the script 126 may present a color slide bar for the health care professional to manipulate to match the patient's pallor. The script 126 may also, in programmed circumstances, direct the nurse to contact a physician, for example, by presenting a “button” for the nurse to select. Alternatively, the script 126 may automatically initiate physician contact, for example, by paging or sending an e-mail. The script 126 may be encoded in a variety of formats such as Java Applets stored at a particular URL (Universal Resource Locator).

The user interface 120 may present other information. For example, the interface 120 can graph collected data, such as, healing progress of an injury or a graph of lung function over time. Additionally, the user interface 120 may provide access (not shown) to reference material for the health care professional conducting the remote session. Further, the user interface 120 may provide links (not shown) to other hospital facilities, for example, to schedule a visit with another health care professional.

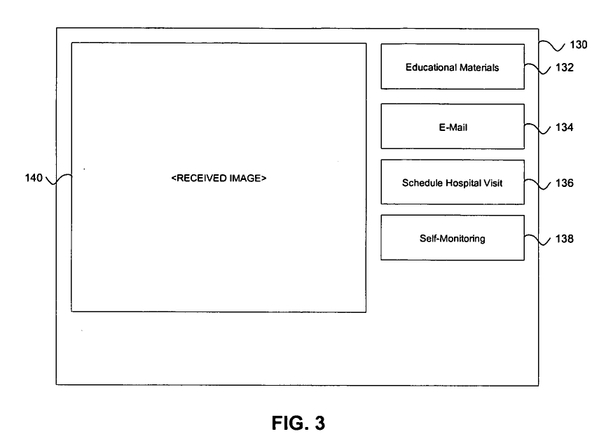

FIG. 3 shows an example of a user interface 130 presented to a patient. As shown, the interface 130 includes a region 140 for presenting images/video received from the health care computer. While not strictly necessary, presenting images of a health care provider can increase a patient's perception of personal attention.

As shown, the patient's user interface 130 also provides access to services that can be accessed even when a remote care session is not in progress. For example, the interface 130 provides access to personally tailored educational materials 132 that can let patients discover answers to common questions at their own pace. The interface 130 can also provide access to an e-mail 134 service that enables patients to e-mail information to a health care provider. For example, a patient can send an e-mail to a doctor or nurse that includes a still image or video of an operation site and the text of a question regarding the image(s). The user interface 130 can also provide access to other hospital systems, for example, to schedule appointments 136, check staff credentials, check prescriptions, and so forth.

The system may also enable a patient to interact with their own treatment plan off-line. For example, the patient's computer may receive computer instructions and/or data from a health care provider that can automatically provide features traditionally provided by human health care providers. For example, the instructions can provide video or text that guides a patient through a data acquisition process. For instance, the instructions may describe and depict a series of steps needed to measure the depth, softness or hardness of an injury and description of pain associated therewith. The instructions may respond to a provided schedule or acquired information (e.g., answers to additional questions, previous measurements, and a doctor's treatment plan encoded in the instructions or data) by suggesting a patient action. In certain instances, the instructions may automatically initiate contact (e.g., page or e-mail) with hospital personnel or instruct the patient to do so.

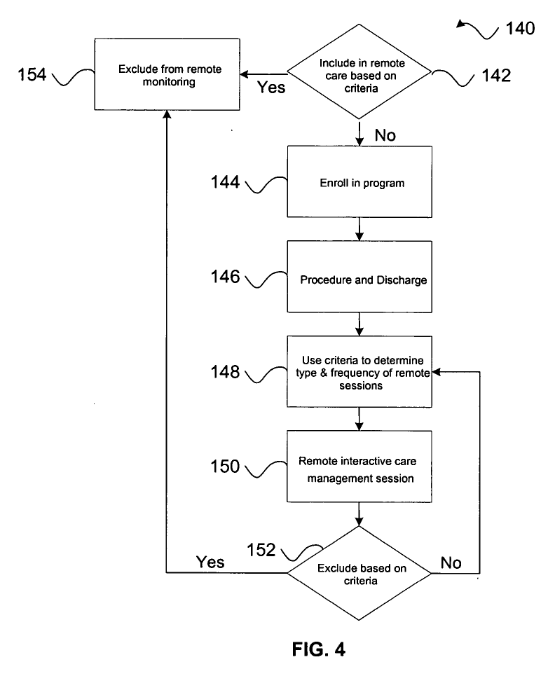

FIG. 4 illustrates a protocol 140 for use with the remote care management system. The protocol 140 helps ensure that remote care does not replace in-person care needed by some patients. The protocol 140 also helps tailor the remote care process to the needs of a particular patient. For example, the protocol 140 can adjust the frequency of remote sessions based on patient characteristics.

The protocol 140 shown is merely exemplary and may vary at different sites and for different purposes. For illustration purposes, this application describes the protocol 140 within the context of a remote monitoring/evaluation protocol 140.

After a normally required hospital admission for a particular medical procedure, patient characteristics are compared 142 to criteria to determine whether remote monitoring/evaluation is appropriate for the patient. Such criteria may include criteria requiring a patient to live within a certain threshold driving distance to a hospital, have a telephone line, have some self-reported or observed familiarity with computers, reside in a home within someone able to assist with physical care, have no co-morbid diseases, a physician referral, and so forth. These criteria are merely examples. Again, these criteria may be removed or altered and others added based on patient satisfaction, outcomes, financial impact, and so forth.

The protocol 140 enrolls 144 patients that meet these criteria and that agree to participate. Enrolled patients receive a computer and instructions, for example, when they come to the hospital for pre-procedure testing. Patients may meet with the nursing staff that will be giving them the post-procedure computer visits. To confirm that they understand the use of the computer, patients receive a trial computer visit prior to their surgery.

After the procedure and discharge 146, patients may receive scheduled remote interactive care management visits 150. For example, the patient may receive an e-mailed schedule identifying times to turn on their computers.

During the remote care management visits 150, nurses, for instance, may use the system to remotely interact with patients and respond in real time. For example, nurses can ask the patients specific questions, examine particular conditions, review care procedures, and so forth, for example, in accordance with the script described in conjunction with FIG. 3. For instance, in response to a patient's comment that a certain area on the body is in pain, a script may suggest asking the patient to describe the severity of the pain and the exact location of the pain. In addition to receiving data from the attached equipment, the nurse can note the patient's appearance as presented by the received video image. Again, the data collected during the interactive visit is stored for subsequent analysis and, potentially, adjustment of protocol 140 criteria.

Enrollment does not limit patient access to more traditional care. For example, patients may call a tele-monitoring nurse or their doctor at any time, request a home visit, and/or schedule an appointment at a hospital. Additionally, even where remote visits form a portion of a patients care, a protocol 140 may schedule both remote and in-person appointments. An in-person post-procedure appointment with a physician may typically be scheduled for 10-14 days after the procedure. Assuming a satisfactory outcome, the patient returns the computer, completes a satisfaction questionnaire, and the patient's participation in the protocol 140 ends.

Through-out the study, the protocol 140 determines 152 whether remote monitoring/evaluation continues to offer an effective method of patient care. Again, the protocol 140 may use different criteria to make this determination 152. For example, the protocol 140 may evaluate a patients vital signs for instability (e.g., a temperature greater than 100, blood pressure less than 90/60 or over 160/100, and/or a pulse greater than 110), evidence of wound bleeding (e.g., conspicuous hematoma or drainage output greater than 100 cc in the first four hours), and/or inadequate pain control as reported and noted by the remote nurse.

The protocol 140 also uses criteria to determine 148 the type and frequency of remote monitoring. For example, the protocol 140 may use patient answers, staff notations, and other collected data to determine a time for the next visit(s). For instance, a slowly recovering patient may be scheduled for a next appointment at an earlier date than a quickly recovering patient.

As described above, in addition to health-based factors, the criteria described above may incorporate resource management considerations. For example, enrollment criteria may depend on the number of nurses trained in use of the system or other resources.

In connection with one embodiment of the present invention, the system 100 may be equipped with certain components to permit, for instance, remote touch-dependent patient evaluation of medical problems. Physicians are typically dependent upon touch to evaluate many medical problems, particularly abdominal symptoms. Physicians often use touch to identify the location where the patient has the most pain. This can lead to identification of the organ that is the cause of such pain. Touch may also be used to identify the severity of, for instance, the abdominal problem. For example if the abdomen is hard, even when the patient is distracted from their abdominal discomfort, this means that the patient has a severe problem in their abdomen. If, on the other hand, the abdomen is soft, it is less likely that there is a significant problem. Physicians may also use touch-dependent examination of the abdomen to detect the presence of a mass that is not otherwise there.

Touch is not only important for abdominal exams, it may be useful in other examination procedures. In particular, breast cancer may be diagnosed by both screening mammogram and physical examination. The physical examination of the breast includes, among other things, observation and palpation or touch. While touching the breast, the physician may be trying to determine whether there is an abnormal mass in the breast that needs further evaluation.

Palpation of an extremity can also lead to determination of the site and severity of an injury. This is particularly true if a patient is complaining of severe pain or decreased function. Exquisite pain solicited by touching a specific site can suggest a bone fracture. Whereas generalized mild to moderate tenderness may be more suggestive of a benign muscle injury.

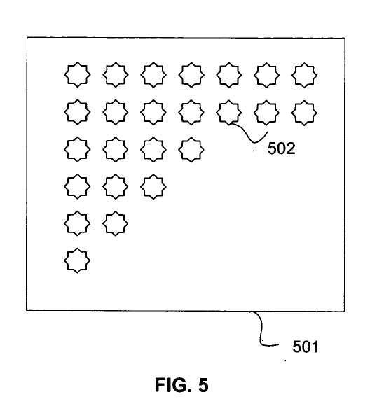

Looking now at FIG. 5, the system 100 may be provided with a pad 501 that is of a size sufficient to cover an area, such as an average abdomen, or at least about one half to one third of an extremity if wrapped around the extremity. In one embodiment, the pad 501 may be, for instance, approximately 14 inches by 12 inches. As the pad 501 may need to conform to the body contour of the patient or wrapped around an extremity, the pad 501 may be made from a malleable material, such as rubber or any flexible material. The pad 501, in accordance with one embodiment of the present invention, may be provided with sensors 502, for instance, pressure sensors, to generate, among other things, a measurement of depth. The sensors 502, may be placed in various configurations, so long as they are capable of measuring depth. In one embodiment, the sensors 502 may be placed within a few millimeters on one another to provide enhanced sensing capability and accuracy.

In use, the pad 501 may be placed over an injured area or an area of discomfort. The sensors 502 on the pad 501 may thereafter be activated remotely by the tending physician to measure characteristics, such as the depth, hardness and/or softness, and location of the injury that may be the source of the pain. The information may thereafter be transmitted in real time to a remote site for evaluation by a physician. Transmission may be by phone or cable line, or wirelessly. It should be noted that the information generated from the touch pad 501 may be used in combination with other remote technologies, including the ones described above, so that a more accurate evaluation can be provided. The touch pad technology can also be used in connection with ultrasound technology, so that the area being touched, i.e., examined, can also be imaged. Additionally, while the pad 501 is on the patient, the patient may be asked to touch the pad 501 covering the injured area. In doing so, the patient may aid in identification of the area of injury, and assist in identify where the pain may be the greatest around the injured area. In one embodiment, the patient can assist by voice correlation, e.g., the louder the voice, the more painful the injured area.

The system described above can used to evaluate many different conditions or for various medical purposes currently employed on an in-patient basis. By employing the use of various add-on components and/or peripherals, the system can be used to promote, for instance, early discharge by offering each preventive care education, monitoring adherence to self-care programs, and gauging patient response to treatment. The system can also be useful for remote wound care monitoring such as chronic leg ulcer management. Frequent monitoring and online reinforcement of self-care instructions can postpone or completely avoid the devastating affect of poorly attended skin trauma. The remote care system can also play an important role in treatment of, for instance, diabetes and reducing in-patient days. For example, individuals with diabetes who have had an imbalance of serum glucose requiring inpatient management but who now have stable chemical results and stable cardio-respiratory status. Patient education, early preventive care, and consistent monitoring are important weapons in preventing many of the devastating vascular consequences of diabetes. The remote care system can also facilitate early discharge for stable maternity patients and offer convenient home care for infants and mothers during the post-partum period.

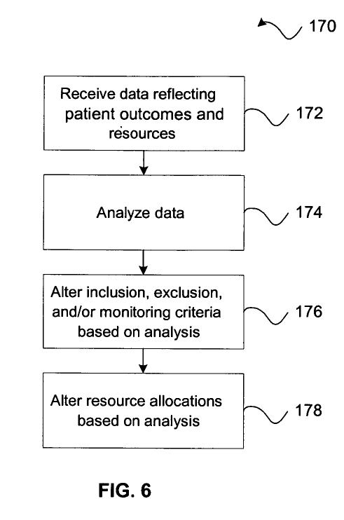

Referring to FIG. 6, the system continually monitors and reacts to the quality and cost of care received by remotely monitored patients. For example, the system may store and statistically analyze data describing patient outcomes, compliance, adverse events, and so forth. The system also monitors costs, charges, and reimbursement of the health care services as well as satisfaction surveys of physicians, payors, and vendors.

Based on this data, the system can modify criteria described above. For example, the system may automatically analyze the data to identify high correlations between criteria parameters and patient satisfaction, outcomes, or data reflecting a high cost. For example, if after time, statistical analysis of data indicates that patients over a certain age do not perform well with remote monitoring, the system may automatically raise the age criteria threshold for continued or initial participation. As another example, the system may identify certain patient conditions requiring more frequent remote sessions and correspondingly alter the protocol's remote session frequency for such patients.

The system may also aggregate data from different sites for comparison and subsequent modification of the protocol criteria. For example, the system may consider analyzing monthly and year-to-date results for aggregated member months, total inpatient costs, inpatient costs, total health provider admissions, admissions by inpatient facility, total inpatient days, inpatient days by health center provider, inpatient days by inpatient facility, and capitation revenue for inpatient care. Additionally, the system may consider average capitation revenue per member per month, average cost for inpatient care, number of admissions per 1000 members per year, number of patient days per 1000 members per year, average length of stay, average cost per day by facility, average cost per admission by facility, average length of stay by inpatient facility. The system may further evaluate on nursing time and activities. Again, based on analysis of this data, the system may automatically adjust the protocol, for example, by altering its criteria.

FIG. 7 depicts a computer 184 suitable for implementing aspects of the techniques described herein. As shown, the computer 184 includes a CPU 186 (Central Processing Unit), volatile memory 188, and non-volatile memory 190. The non-volatile memory 190 can store instructions 192 for implementing a protocol. The non-volatile memory 190 may also include instructions 196 for adjusting the protocol in response to collected data. Such instructions 196 may include instructions for statistically analyzing patient data 198 or other collected data. In the course of operation, the instructions 192, 196 are transferred from the non-volatile memory 190 to the volatile memory 188 and/or the CPU 186 for execution.

As shown, the computer 184 may also store protocol criteria and logic 194. The protocol logic 194 may be encoded using any of a variety of computer languages. The computer 184 may also store other information such as scripts (not shown) for use by health care professionals during a remote session and instructions that enable a user to access their treatment plan off-line.

As shown, the computer also features a network connection 182. As such, the features described above may be distributed across many different computers. For example, one computer may store patient data while another stores scripts for transmission to care taker computers.

The techniques described herein, however, are not limited to any particular hardware or software configuration. The techniques may be implemented in hardware or software, or a combination of the two. Preferably, the techniques are implemented in computer programs executing on programmable computers that each include a processor, a storage medium readable by the processor (including volatile and non-volatile memory and/or storage elements), at least one input device, and one or more output devices.

Each program is preferably implemented in high level procedural or object oriented programming language to communicate with a computer system. However, the programs can be implemented in assembly or machine language, if desired. In any case the language may be compiled or interpreted language.

Each such computer program is preferably stored on a storage medium or device (e.g., CD-ROM, hard disk, or magnetic disk) that is readable by a general or special purpose programmable computer for configuring and operating the computer when the storage medium or device is read by the computer to perform the procedures described herein. The system may also be considered to be implemented as a computer-readable storage medium, configured with a computer program, where the storage medium so configured causes a computer to operate in a specific and predefined manner.

While the invention has been described in connection with the specific embodiments thereof, it will be understood that it is capable of further modification. Furthermore, this application is intended to cover any variations, uses, or adaptations of the invention, including such departures from the present disclosure as come within known or customary practice in the art to which the invention pertains.

1. A system for use in remote touch-dependent evaluation, the system comprising:
a computer;
a pad sufficiently sized to at least partially cover an area to be evaluated and in communication with the computer;
a plurality of sensors placed on the pad to measure characteristics of the area to be evaluated; and
data transmission means connected to a public network for transmitting, in real time, measured characteristics to a remote location for evaluation.

2. A system as set forth in claim 1, wherein the sensors are positioned relatively close to one another to provide enhanced sensing capability and accuracy.

3. A method for remote touch-dependent evaluation, the method comprising:
providing a pad sufficiently sized to at least partially cover an area to be evaluated on a patient and having a plurality of sensors to measure characteristics of the area to be evaluated;
positioning the pad on to a body part to be evaluated;
activating the sensors on the pad to initiate measurement;
transmitting, in real time, measured characteristics to a remote site; and
reviewing, from the remote site, the measured characteristics for evaluation purposes.

4. A method as set forth in claim 3, wherein the step of activating includes remotely activating the sensor from the remote site.

5. A method as set forth in claim 3, further including having the patient identify the area to be evaluated and characteristics about the area.
